(19) United States
(12) Patent Application Publication
(10) Pub. No.: US 2023/0397892 A1
(43) Pub. Date: Dec. 14, 2023

(54) DETECTING USER INFECTION USING WEARABLE SENSOR DATA

(71) Applicant: KONINKLIJKE PHILIPS N.V., ENDHOVEN (NL)

(72) Inventors: Ikaro Garcia Araujo da Silva, Cambridge, MA (US); Bryan Conroy, Cambridge, MA (US); Sara Mariani, Cambridge, MA (US)

(21) Appl. No.: 18/209,007

(22) Filed: Jun. 13, 2023

Related U.S. Application Data
(60) Provisional application No. 63/351,516, filed on Jun. 13, 2022.

(51) Int. Cl. A61B 5/00; A61B 5/024; A61B 5/318

(52) U.S. Cl. CPC A61B 5/7275; A61B 5/02405; A61B 5/742; A61B 5/4836; A61B 5/318; A61B 5/6802

(57) ABSTRACT

A method for reporting a user's risk of infection, comprising: (i) receiving, from a sensor of a wearable device worn by the user, user heart data; (ii) determining, from the received user heart data by a pulse rate variability phase acceleration algorithm, the user's pulse rate variability phase acceleration values for the first time period; (iii) determining, from the received user heart data by a pulse rate variability phase deceleration algorithm, the user's pulse rate variability phase deceleration values for the first time period; (iv) determining, by a trained infection risk prediction algorithm using the user's pulse rate variability phase acceleration values and the user's pulse rate variability phase deceleration values for the first time period, the user's risk of a current infection; and (v) providing, via a user interface, the determined user's risk of a current infection.

### CROSS-REFERENCE TO RELATED APPLICATIONS

This patent application claims the priority benefit under 35 U.S.C. § 119(e) of U.S. Provisional Application No. 63/351,516, filed on Jun. 13, 2022, the contents of which are herein incorporated by reference.

### BACKGROUND OF THE INVENTION

### 1. Field of the Invention

The present disclosure is directed generally to methods and systems for detecting, reporting, and/or treating an individual's infection as detected by wearable device sensor data.

### 2. Description of the Related Art

Detection of infection is an important aspect of personal health care. Early detection of infection leads to both improved treatment, and also improves public health from an epidemiological viewpoint. However, detecting an individual's infection outside of a care facility is challenging, especially if the individual is not experiencing significant symptoms or does not otherwise recognize that they may have an infection. Although there are many ways for a user to actively monitor their own health and detect an infection, passive monitoring or detection systems that detect infection without requiring active input by the user would vastly improve early infection detection as well as public health monitoring, including but not limited to nosocomial infections due to hospital staff transmissions, or outbreaks in military aircraft carriers, among other scenarios.

### SUMMARY OF THE INVENTION

Accordingly, there is a continued need for methods and systems that detect patient infection or a risk of infection using a variety of data inputs, especially via passive monitoring. Various embodiments and implementations herein are directed to a method and system configured to detect and report patient infection or risk of infection based on sensor data from a wearable device worn by the patient. A system receives user heart data from a sensor of a wearable device worn by the user. The system then determines, from the received user heart data, the user's pulse rate for a first time period. The system determines from the received user heart data, both: (i) the user's pulse rate variability phase acceleration values for the first time period by a pulse rate variability phase acceleration algorithm; and (ii) the user's pulse rate variability phase acceleration values for the first time period by a pulse rate variability phase deceleration algorithm. A trained infection risk prediction algorithm of the system determines, using the user's pulse rate variability phase acceleration values and the user's pulse rate variability phase deceleration values for the first time period, the user's risk of a current infection. The determined user's risk of a current infection is provided via a user interface. According to an embodiment, a medical professional prescribes an antibiotic and/or anti-viral treatment to treat the user's current infection in response to receiving the provided determined user's risk of a current infection.

Generally, in one aspect, a method for reporting a user's risk of infection is provided. The method includes: (i) receiving, from a sensor of a wearable device worn by the user, user heart data; (ii) determining, from the received user heart data by a pulse rate variability phase acceleration algorithm, the user's pulse rate variability phase acceleration values for the first time period; (iii) determining, from the received user heart data by a pulse rate variability phase deceleration algorithm, the user's pulse rate variability phase deceleration values for the first time period; (iv) determining, by a trained infection risk prediction algorithm using the user's pulse rate variability phase acceleration values and the user's pulse rate variability phase deceleration values for the first time period, the user's risk of a current infection; and (v) providing, via a user interface, the determined user's risk of a current infection.

According to an embodiment, the method further includes prescribing, by a medical professional in response to the provided determined user's risk of a current infection, an antibiotic and/or anti-viral treatment to treat the user's current infection.

According to an embodiment, the method further includes transmitting the received user heart data to a remote server, wherein said determining steps are performed at the remote server.

According to an embodiment, the user interface comprises a web portal.

According to an embodiment, the method further includes receiving, by a medical professional via the user interface, determined user's risk of a current infection.

According to an embodiment, the method is performed at a regular interval for the user by a remote service.

According to an embodiment: (i) the sensor is a heart rate sensor, and the user heart data is heart rate sensor data, and/or (ii) the sensor is an electrocardiography sensor, and the user heart data is ECG data.

Also provided is a method for treating a patient's infection. The method includes receiving, by a medical professional from a user interface, a patient's determined current infection, wherein the patient's current infection is determined by: (i) receiving, from a sensor of a wearable device worn by the patient, patient heart data; (ii) determining, from the received patient heart data by a pulse rate variability phase acceleration algorithm, the patient's pulse rate variability phase acceleration values for the first time period; (iii) determining, from the received patient heart data by a pulse rate variability phase deceleration algorithm, the patient's pulse rate variability phase deceleration values for the first time period; (iv) determining, by a trained infection risk prediction algorithm using the patient's pulse rate variability phase acceleration values and the patient's pulse rate variability phase deceleration values for the first time period, that the patient is experiencing a current infection; and (v) providing, to the medical professional via the user interface, the determination that the patient is experiencing a current infection. The method also includes prescribing, by the medical professional in response to the provided patient's risk of a current infection, an antibiotic and/or anti-viral treatment to treat the patient's current infection.

Also provided is a system for reporting a user's predicted risk of infection. The system includes a wearable device worn by the user, comprising a sensor configured to obtain user heart data; a trained infection risk prediction algorithm configured to determine the user's risk of a current infection based on the user's pulse rate variability phase acceleration values and the user's pulse rate variability phase deceleration values for the first time period; a processor configured to: (i) determine, from the received user heart data by a pulse rate variability phase acceleration algorithm, the user's pulse rate variability phase acceleration values for the first time period; (ii) determine, from the received user heart data by a pulse rate variability phase deceleration algorithm, the user's pulse rate variability phase deceleration values for the first time period; and (iii) determine, by the trained infection risk prediction algorithm using the user's pulse rate variability phase acceleration values and the user's pulse rate variability phase deceleration values for the first time period, the user's risk of a current infection; and a user interface configured to provide the determined user's risk of a current infection, as well as population based assessments and rankings such that personnel responsible for infections control can efficiently monitor a cohort and respond to outbreaks.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein. These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for reporting a user's predicted risk of infection, in accordance with an embodiment;

FIG. 2 is a schematic representation of an infection detection system, in accordance with an embodiment;

FIG. 3 is a flowchart of a method for reporting a user's predicted risk of infection, in accordance with an embodiment; and

FIG. 4 is a flowchart of a method for training an infection prediction algorithm, in accordance with an embodiment.

### DETAILED DESCRIPTION OF EXEMPLARY EMBODIMENTS

The present disclosure describes various embodiments of a system and method configured to detect an individual's infection and/or to determine an individual's risk or likelihood of having a current infection. More generally, Applicant has recognized and appreciated that it would be beneficial to provide a method or system to improve infection detection using heart rate data, obtained from a sensor of a wearable device. Accordingly, an infection detection system receives user heart data from a sensor of a wearable device worn by the user. The system then determines, from the received user heart data, the user's pulse rate for a first time period. The system determines from the received user heart data, both: (i) the user's pulse rate variability phase acceleration values for the first time period by a pulse rate variability phase acceleration algorithm; and (ii) the user's pulse rate variability phase acceleration values for the first time period by a pulse rate variability phase deceleration algorithm. A trained infection risk prediction algorithm of the system determines, using the user's pulse rate variability phase acceleration values and the user's pulse rate variability phase deceleration values for the first time period, the user's risk of a current infection. The determined user's risk of a current infection is provided via a user interface. According to an embodiment, a medical professional prescribes an antibiotic and/or anti-viral treatment to treat the user's current infection in response to receiving the provided determined user's risk of a current infection.

According to an embodiment, the systems and methods described or otherwise envisioned herein can, in some non-limiting embodiments, be implemented as an element for a commercial product for patient analysis or monitoring, such as Philips® tele-health products, Connected Care platforms, HealthBand, and/or HealthDot® (available from Koninklijke Philips NV, the Netherlands), or any suitable system.

According to another embodiment, the systems and methods described or otherwise envisioned herein can, in some non-limiting embodiments, be implemented as an element for cohort monitoring. For example, the systems and methods could be utilized by system by infection control in a hospital to monitor staff and/or patients, in order to minimize the rate of nosocomial infections. As another example, the systems and methods could be utilized by system by institutions such as the military or higher education, which are responsible for public safety of their community, so they can monitor and respond to potential outbreaks. Many other embodiments are possible.

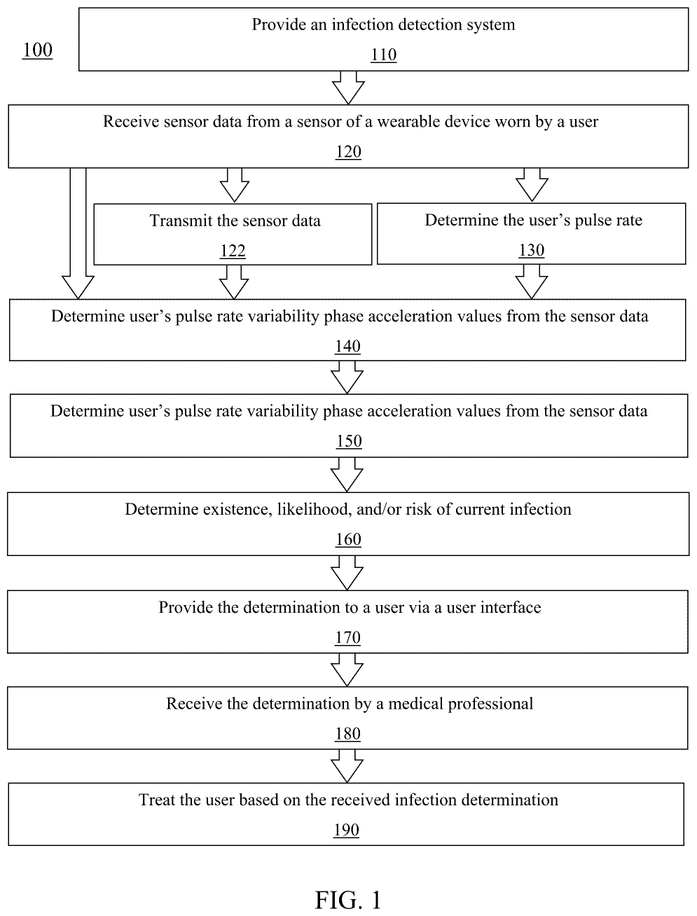

Referring to FIG. 1, in one embodiment, is a flowchart of a method 100 for detecting and reporting an individual's infection or likelihood of infection using an infection detection system. The methods described in connection with the figures are provided as examples only, and shall be understood not limit the scope of the disclosure. The infection detection system can be any of the systems described or otherwise envisioned herein. The infection detection system can be a single system or multiple different systems.

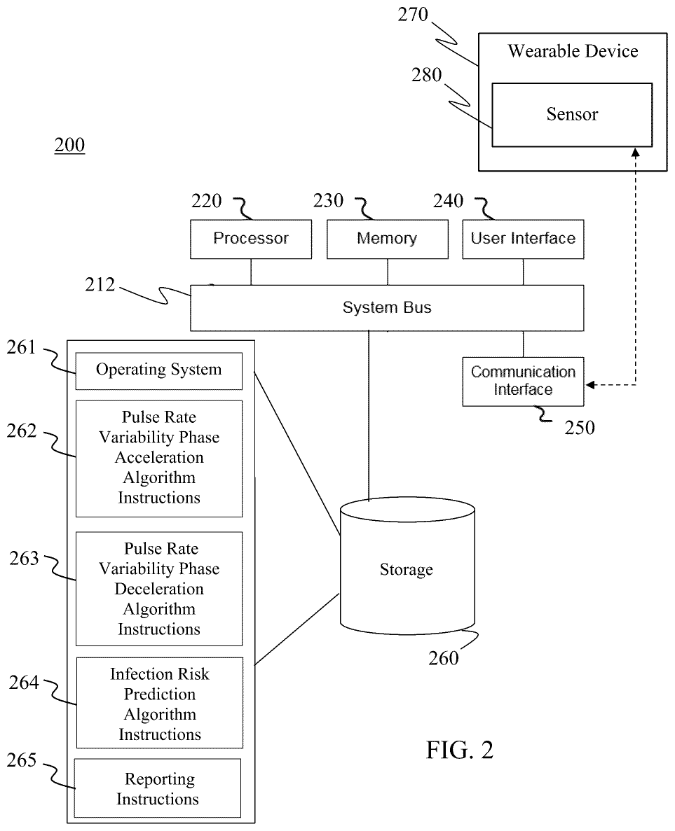

At step 110 of the method, an infection detection system is provided. Referring to an embodiment of an infection detection system 200 as depicted in FIG. 2, for example, the system comprises one or more of a processor 220, memory 230, user interface 240, storage 260, wearable device 270 with sensor 280, interconnected via one or more system buses 212 and/or via one or more communications interfaces 250. It will be understood that FIG. 2 constitutes, in some respects, an abstraction and that the actual organization of the components of system 200 may be different and more complex than illustrated. Additionally, infection detection system 200 can be any of the systems described or otherwise envisioned herein. Other elements and components of infection detection system 200 are disclosed and/or envisioned elsewhere herein. According to an embodiment, the infection detection system 200 comprises a wearable device or other sensor device in communication with a local or remote processor, such as a cloud-based processing system.

According to an embodiment, a user wears a wearable device that comprises a sensor configured to obtain sensor data about the user. The wearable device may be the infection detection system 200, or the wearable device may be a component of the infection detection system 200. The user may be any individual capable of wearing the wearable device. According to an embodiment, the user is a patient. For example, a patient may be discharged with or otherwise given a wearable device or a prescription or order to purchase and/or use the wearable device. As another example, the user may be a member of the military or any other organization for which monitoring the health of the user is advantageous. As yet another example, the user may be any individual interested in monitoring their health.

The infection detection system 200 may be offered as a software as a service (SAAS), in which a wearable device is sold to a user along with, or with an offer to use or purchase, the service. For example, a user may buy a wearable device to monitor one aspect of their health such as activity levels, or to provide other monitoring or notification services. That wearable device may be accompanied with user access to the infection detection system 200, as a free or paid service.

The wearable device may be any device that can be worn or carried or otherwise in communication with the user such that the sensor of the device can obtain the sensor data about the user. The wearable device may be a ring, a watch, a necklace, or any other device. Examples of wearable devices include the Apple® watch, Garmin® watch, Oura® ring, Empatica® bracelet, and many, many more. These devices typically monitor activity levels and/or provide notifications or other communication options, and can monitor vital signals that typically include: photoplethysmography (PPG), skin temperature, acceleration, oxygen saturation, and other vital signs.

At step 120 of the method, the infection detection system 200 receives sensor data from a sensor 210 of the wearable device 270. According to an embodiment, the wearable device is the infection detection system and thus a processor of the wearable device receives sensor data from a sensor of the wearable device. According to another embodiment, the wearable device is a component of the infection detection system and a local and/or remote processor of the infection detection system—such as a remote server—receives sensor data from the sensor transmitted by wired and/or wireless communication.

According to an embodiment, the sensor data may be any data from which the input for the method and system described or otherwise envisioned herein can utilize to determine the likelihood, risk, or presence of an infection. For example, the sensor data may be an electrocardiogram obtained from one or more electrode sensors. The sensor data may be heartbeat or rate data from a photoplethysmography (PPG) sensor or other heart sensor. Other sensors may be suitable.

According to an embodiment, sensor data may be obtained or received by the infection detection system continually, or it may be obtained or received periodically such as at a predetermined request rate. For example, sensor data may be utilized immediately or may be stored in local or remote storage for use in further steps of the method. Thus, the infection detection system can receive sensor data from, or request sensor data from, the sensor and/or storage continually or at a predetermined request rate. For example, at optional step 122 of the method, the sensor data and/or other data is transmitted to a remote server or other component of the infection detection system.

At step 130 of the method, a processor of the infection detection system analyzes the received sensor data to determine the user's pulse rate for a first time period. The first time period may be any time period for which sufficient sensor data may be obtained and analyzed by the system. For example, in a scenario where the infection detection system analyzes the data continually, the first time period may be a minute, less than a minute, or several/many minutes, and the first time period and a subsequent time period may or may not overlap. In a scenario where the infection detection system analyzes the data periodically, the first time period may be any selected period of time, such as a minute, less than a minute, or several/many minutes. The user's pulse rate for the first time period may be determined using any method, algorithm, or process for extracting or otherwise calculating or determining pulse rate from the received sensor data type.

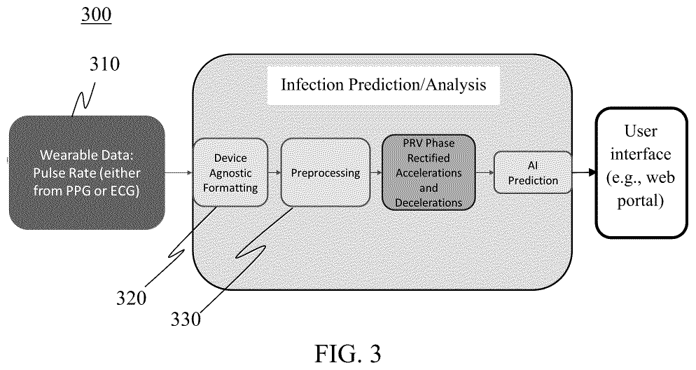

According to an embodiment, before or after step 130, or at any other point in the method, the infection detection system may prepare the received sensor data for analysis. For example, Referring to FIG. 3, is a flowchart of a method for infection detection or prediction. At 310, the sensor data is obtained by a sensor of the wearable device, and is transmitted to, received by, or otherwise utilized by the infection detection system (which may be the wearable device and/or other components of a system) to detect or predict infection. At 320 and 330, the received sensor data is prepared for analysis. For example, at 320, the sensor data is formatted for processing. Since the infection detection system can be configured to utilize sensor data from a wide variety of different wearable devices, the format of the sensor data may vary from wearable device to wearable device. Accordingly, the infection detection system can be configured, designed, programmed, or otherwise set up to process sensor data of two or more different types, such as from two or more different wearable device types or brands, to generate a standardized sensor data that is utilized by downstream steps of the method. This may include removing extraneous sensor data, reformatting or otherwise reorganizing sensor data, and other processing steps.

At step 330, the received sensor data (which may or may not have required formatting or reformatting at 320) can be pre-processed such that it can easily be utilized by downstream steps of the method. Pre-processing can comprise any data analysis or modification required to change the sensor data into suitable input data. For example, pre-processing can include cleaning the data such as removing noise or outliers, such as replacing the top and bottom 1% of data using random uniform values generated from the neighboring data, among many, many other options.

The formatted and processed sensor data may be utilized immediately or may be stored in local or remote storage of the infection detection system, such as in a memory of the wearable device or other component of the system, for use in further steps of the method.

At step 140 of the method, a processor of the infection detection system uses a pulse rate variability phase acceleration algorithm to analyze the received sensor data in order determine the user's pulse rate variability phase acceleration values for the first time period. The pulse rate variability phase acceleration algorithm is any algorithm configured to analyze the received sensor data, and generate phase rectified heart rate acceleration data. For example, the pulse rate variability phase acceleration algorithm averages out noise that affects the heart rate acceleration. According to an embodiment, from the sensor data, including inter-beat intervals, information such as pulse rate can be obtained, along with the pulse rate variability. Since heart rate and heart rate variability can be altered with infection, the heart rate and heart rate variability can be translated into pulse rate variability for processing by the system.

The user's pulse rate variability phase acceleration values for the first time period may be utilized immediately or may be stored in local or remote storage of the infection detection system, such as in a memory of the wearable device or other component of the system, for use in further steps of the method.

At step 150 of the method, a processor of the infection detection system uses a pulse rate variability phase deceleration algorithm to analyze the received sensor data in order determine the user's pulse rate variability phase deceleration values for the first time period. The pulse rate variability phase deceleration algorithm is any algorithm configured to analyze the received sensor data, and generate phase rectified heart rate deceleration data. For example, the pulse rate variability phase deceleration algorithm averages out noise that affects the heart rate deceleration. According to an embodiment, from the sensor data, including inter-beat intervals, information such as pulse rate can be obtained, along with the pulse rate variability. Since heart rate and heart rate variability can be altered with infection, the heart rate and heart rate variability can be translated into pulse rate variability for processing by the system.

The user's pulse rate variability phase deceleration values for the first time period may be utilized immediately or may be stored in local or remote storage of the infection detection system, such as in a memory of the wearable device or other component of the system, for use in further steps of the method.

According to an embodiment, the pulse rate variability phase acceleration algorithm and/or the pulse rate variability phase deceleration algorithm can be algorithms from, based on, or otherwise derived from, algorithms described in Kamath et al. (Eds.), “Heart Rate Variability (HRV) Signal Analysis: Clinical Applications,” (2012). However, other pulse rate variability phase acceleration and/or deceleration algorithms are possible.

At step 160 of the method, a trained infection risk prediction algorithm of the infection detection system utilizes at least the user's pulse rate variability phase acceleration values and the user's pulse rate variability phase deceleration values for the first time period as input to generate a determination of current infection and/or a risk or likelihood of current infection. The infection risk prediction algorithm of the infection detection system can be any algorithm configured to utilize these inputs and generate a determination of current infection and/or a risk or likelihood of current infection. For example, the algorithm can be a machine learning algorithm of any kind, as well as other types of algorithms. The algorithm can be trained using known mechanisms for training machine learning algorithms.

According to an embodiment, the infection detection system utilizes the trained infection risk prediction algorithm to determine the user's current infection and/or a risk or likelihood of current infection according to the follow process. However, this is provided as a non-limiting example only, and other methods may be utilized to determine the user's current infection and/or a risk or likelihood of current infection with the algorithm.

According to this embodiment, the infection detection system receives sensor data, such as inter-beat-intervals (IBI) from the sensor for a first time period, such as an entire sleep episode (e.g., eight hours). The infection detection system breaks the sleep episode IBI into 5-minute windows, or into windows of any other size. The size of the windows can be a predetermined size, or can be a learned size.

For each window, the infection detection system computes pulse rate variability phase acceleration values and pulse rate variability phase deceleration values using the sensor data. According to an embodiment, to generate pulse rate variability phase acceleration values (and similarly to generate pulse rate variability phase deceleration values), the system creates a buffer, called anchors, to store all beats that are increased (decreased) in value. This buffer horizontally stacks all beats that result in increased heart rate. The dimensions of this buffer are N×L, where N is the number of beats that resulted in increased (decreased) heart rate. L is the window length, which means that the system will store all the IBI values of L/2 beats before the selected beat, and L/2 beats after the selected beat of row N. According to one embodiment, L=4, so the system is storing two IBI values before, the current IBI value, and one IBI value after the current beat.

According to an embodiment, for each beat in the window, if the IBI increases in value (or decreases in value, during analysis by the deceleration algorithm), the system add the window's IBI to the anchors buffer along with the IBI of the previous and following beats. For optional filtering, if the IBI varies by more than 5%, the system can reject the beat and not enter it into the anchors buffer.

According to an embodiment, if there are at least 20 beats stored in the anchors buffer (i.e., N≥20), the system computes the average across all the anchors beats by calculating the mean across the rows. This will result in the anchors buffer now having dimensions 1×4. According to an embodiment, the resulting phase rectified acceleration and decelerations for the window period (5 minutes, in this non-limiting example) is given by the average difference of the across the pairs of beats before and after the fiducial beat, according to the following equation:

output=(average[2]+average[3]−average[0]−average[1]/4) (Eq. 1.)

According to an embodiment, the infection detection system aggregates all the window values into single daily statistics, including but not limited to minimum, mean, maximum, standard deviation, quantiles, and more.

According to an embodiment, the infection detection system aggregates all information across multiple times (such as across 10 days, according to one non-limiting example), stacks them into a vector, and inputs that information into the machine learning algorithm to generate an infection prediction score or determination.

The infection prediction score or determination can be a binary score indicating infection (e.g., yes/no, I/O), a risk of infection (e.g., low risk, moderate risk, high risk), and/or prediction of infection (e.g., such as a percentage likelihood of infection), or any other indication of the user's infection, risk of infection, or likelihood of infection.

According to an embodiment, the infection prediction score or determination may be utilized immediately or may be stored in local or remote storage of the infection detection system, such as in a memory of the wearable device or other component of the system, for use in further steps of the method.

At step 170 of the method, the determined infection, infection score, risk of infection, and/or prediction of infection is provided via a user interface of the infection detection system. The system may display or otherwise provide the information to a user, such as the wearer of the wearable device, a care provider, or other individual, via the user interface. The display may comprise information about the patient, the input data for the patient, or any other information. The infection information may be communicated by wired and/or wireless communication to another device. For example, the system may communicate the infection information and other information to a mobile phone, computer, laptop, wearable device, and/or any other device configured to allow display and/or other communication of the infection information and other information. The user interface can be any device or system that allows information to be conveyed and/or received, and may include a display, a mouse, and/or a keyboard for receiving user commands. According to an embodiment, the user interface is a web portal that the user or other individual accesses to obtain the infection information and/or other information.

According to an embodiment, the user interface may comprise an alert system. For example, the system or user interface may be configured to provide or otherwise generate an alert if the infection detection system detects infection, or detects a risk or likelihood of infection that meets or exceeds a predetermined threshold. A moderate risk or >50% likelihood of infection may trigger the system or user interface to generate an alert. An alert may be, for example, a noise, haptic response, text message, or any other mechanism of alerting a user or other individual to information.

At optional step 180 of the method, a medical professional or other professional user or individual receives the determined infection, risk of infection, or likelihood of infection for the wearer of the wearable device via the user interface. The medical professional or other professional user or individual can be anyone that will utilize the provided information for a management or care decision. For example, the individual can be the wearer's physician. As another example, the individual can be a manager of an organization or other group that is monitoring one or more wearers for potential infection.

According to an embodiment, the medical professional or other professional user or individual can receive the determined infection, risk of infection, or likelihood of infection via the user interface using any mechanism. For example, the individual may receive an alert or other information indicating the determined infection, risk of infection, or likelihood of infection. Alternatively, the user may log into a web portal to receive or otherwise access the information. As yet another option, the user may have an app installed on their smartphone or other device to access the information, and that app may further comprise a notification or alert mechanism.

At optional step 190 of the method, the medical professional or other professional user or individual has received the information and can act on that information. For example, a medical professional may receive and review the determined infection, risk of infection, or likelihood of infection for the wearer of the wearable device. After reviewing the information, the medical professional can make a treatment or therapy decision. Since the infection detection system is identifying infection or risk of infection, the treatment or therapy is designed or selected to treat that infection or prevent infection. For example, the treatment may comprise an antibiotic to treat an infection from a bacterial agent, the treatment may comprise an antiviral to treat an infection from a viral agent, and/or the treatment may comprise an anti-fungal to treat an infection from a fungal agent, the treatment may comprise an anti-parasitic to treat an infection from a parasitic agent, among other possible treatments.

The selected antibiotic, antiviral, antifungal, or antiparasitic may be selected to broadly treat infection, or may be more narrowly selected to treat a very specific bacterial agent, virus, fungus, or parasite. The selected antibiotic, antiviral, antifungal, or antiparasitic may also be based on information about the wearer of the wearable device, including but not limited recent travel history, exposure history, medical history, demographic information, and/or any other information. Since any or all of this information may be stored by or otherwise accessible to the infection detection system, all of this information may be available to the medical professional and therefore can inform the selected treatment or therapy. The selected treatment or therapy can be communicated directly or indirectly to the wearer of the wearable device, such as through an electronic communication, a prescription, a phone call, a voicemail, a text message, or any other mechanism.

Referring to FIG. 2 is a schematic representation of an infection detection system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, system 200 comprises a wearable device 270 with one or more sensors 280 configured to obtain a sensor signal or sensor data about the patient, namely regarding heartbeat data about the patient. The wearable device can be any device capable of comprising the sensor and obtaining the sensor data, including but not limited to a ring, watch, bracelet, necklace, clothing, and other devices. Although shown in FIG. 2 as a component of system 200, system 200 may be components of the wearable device 270. The sensor 280 may be any sensor capable of generate the sensor data described or otherwise envisioned herein.

According to an embodiment, storage 260 of system 200 may store one or more algorithms, modules, and/or instructions to carry out one or more functions or steps of the methods described or otherwise envisioned herein. For example, the system may comprise, among other instructions or data, pulse rate variability phase acceleration algorithm instructions 262, pulse rate variability phase deceleration algorithm instructions 263, trained infection risk prediction algorithm 264, and reporting instructions 265.

According to an embodiment, the pulse rate variability phase acceleration algorithm instructions 262 direct the system to analyze the received sensor data in order determine the user's pulse rate variability phase acceleration values for the first time period. The pulse rate variability phase acceleration algorithm is any algorithm configured to analyze the received sensor data, and generate phase rectified heart rate acceleration data. For example, the pulse rate variability phase acceleration algorithm averages out noise that affects the heart rate acceleration. The user's pulse rate variability phase acceleration values for the first time period may be utilized immediately or may be stored in local or remote storage of the infection detection system, such as in a memory of the wearable device or other component of the system, for use in further steps of the method.

According to an embodiment, the pulse rate variability phase deceleration algorithm instructions 263 direct the system to analyze the received sensor data in order determine the user's pulse rate variability phase deceleration values for the first time period. The pulse rate variability phase deceleration algorithm is any algorithm configured to analyze the received sensor data, and generate phase rectified heart rate deceleration data. For example, the pulse rate variability phase deceleration algorithm averages out noise that affects the heart rate deceleration. The user's pulse rate variability phase deceleration values for the first time period may be utilized immediately or may be stored in local or remote storage of the infection detection system, such as in a memory of the wearable device or other component of the system, for use in further steps of the method.

According to an embodiment, the trained infection risk prediction algorithm 264 directs the system to utilize at least the user's pulse rate variability phase acceleration values and the user's pulse rate variability phase deceleration values for the first time period as input to generate a determination of current infection and/or a risk or likelihood of current infection. The infection risk prediction algorithm of the infection detection system can be any algorithm configured to utilize these inputs and generate a determination of current infection and/or a risk or likelihood of current infection. For example, the algorithm can be a machine learning algorithm of any kind, as well as other types of algorithms. The algorithm can be trained using known mechanisms for training machine learning algorithms.

According to an embodiment, the reporting instructions 265 direct the system to provide the determined infection, infection score, risk of infection, and/or prediction of infection to a user via a user interface of the infection determination system. The system may display or otherwise provide the infection information to a user, such as a care provider or the wearer of the wearable device, via the user interface. The infection information may comprise the determined infection, infection score, risk of infection, and/or prediction of infection as well as other information such as information about the wearer of the wearable device. The information may be communicated by wired and/or wireless communication to another device. For example, the system may communicate the information to a mobile phone, computer, laptop, wearable device, and/or any other device configured to allow display and/or other communication of the directive and other information. The user interface can be any device or system that allows information to be conveyed and/or received, and may include a display, a mouse, and/or a keyboard for receiving user commands.

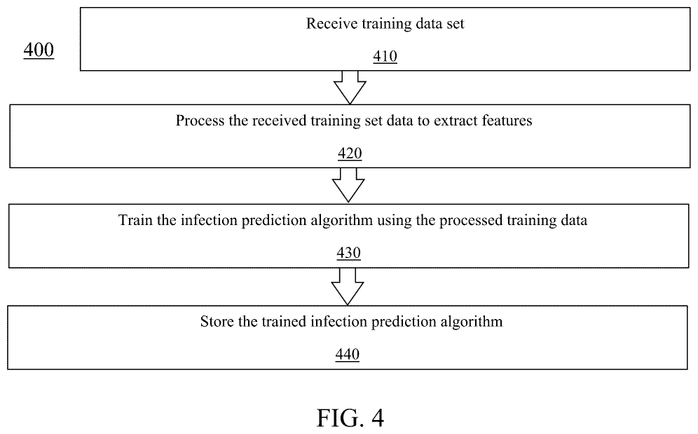

Referring to FIG. 4, in one embodiment, is a flowchart of a method 400 for training the infection prediction algorithm 264 of the infection detection system 200. At step 410 of the method, the system receives a training data set comprising heartbeat data and infection status data for a plurality of individuals. The training data can comprise any information or data necessary to train the infection prediction algorithm, including labeled heartbeat data and infection status data, indicating the absence or presence of infection. The training data may be stored in and/or received from one or more databases. The database may be a local and/or remote database. For example, the infection detection system may comprise a database of training data.

According to an embodiment, the infection detection system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

At step 420 of the method, the system processes the received information to extract localization features about the heartbeat and infection status data. The heartbeat and infection status features may be any features which will be utilized to train the infection prediction algorithm, such as any heartbeat and infection status features that can or will be utilized by the trained algorithm for infection analysis for a future heartbeat data. Feature extraction can be accomplished by a variety of embodiments for feature identification, extraction, and/or processing, including any method for extracting features from a dataset. The outcome of a feature processing step or module of the ultrasound system is a set of heartbeat and infection status features about a plurality of wearers of wearable devices, which thus comprises a training data set that can be utilized to train the classifier.

At step 430 of the method, the system trains the machine learning algorithm, which will be the algorithm utilized in analyzing heartbeat information as described or otherwise envisioned. The machine learning algorithm is trained using the extracted features according to known methods for training a machine learning algorithm. According to an embodiment, the algorithm is trained, using the processed training dataset, to generate a determined infection, infection score, risk of infection, and/or prediction of infection, as described or otherwise envisioned herein. The generated information or report can comprise any of the information described or otherwise envisioned herein, including but not limited to a determined infection, infection score, risk of infection, and/or prediction of infection for the wearer of a wearable device.

At step 440 of the method, the trained infection prediction algorithm is stored for future use. According to an embodiment, the trained model may be stored in local or remote storage.

According to an embodiment, the infection detection system is configured to process many thousands or millions of datapoints in the input data used to train the classifier, as well as to process and analyze the received plurality of heartbeat and infection status features. For example, generating a functional and skilled trained classifier using an automated process such as feature identification and extraction and subsequent training requires processing of millions of datapoints from input data and the generated features. This can require millions or billions of calculations to generate a novel trained classifier from those millions of datapoints and millions or billions of calculations. As a result, each trained classifier is novel and distinct based on the input data and parameters of the machine learning algorithm, and thus improves the functioning of the infection detection system. Thus, generating a functional and skilled trained classifier comprises a process with a volume of calculation and analysis that a human brain cannot accomplish in a lifetime, or multiple lifetimes.

In addition, the infection detection system can be configured to continually receive heartbeat and infection status features, perform the analysis, and provide periodic or continual updates via the report provided to a user for the wearer of the wearable device. This requires the analysis of thousands or millions of datapoints on a continual basis to optimize the reporting, requiring a volume of calculation and analysis that a human brain cannot accomplish in a lifetime.

By providing improved infection prediction or detection, this novel infection detection system has an enormous positive effect on patient care compared to prior art systems. As just one example, by providing a system that can improve user analysis by detecting infection at early stages, the system can facilitate early treatment decisions and improve survival outcomes, thereby leading to saved lives.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for reporting a user's risk of infection, comprising:
receiving, from a sensor of a wearable device worn by the user, user heart data;
determining, from the received user heart data by a pulse rate variability phase acceleration algorithm, the user's pulse rate variability phase acceleration values for the first time period;
determining, from the received user heart data by a pulse rate variability phase deceleration algorithm, the user's pulse rate variability phase deceleration values for the first time period;
determining, by a trained infection risk prediction algorithm using the user's pulse rate variability phase acceleration values and the user's pulse rate variability phase deceleration values for the first time period, the user's risk of a current infection; and
providing, via a user interface, the determined user's risk of a current infection.

2. The method of claim 1, further comprising the step of prescribing, by a medical professional in response to the provided determined user's risk of a current infection, an antibiotic and/or anti-viral treatment to treat the user's current infection.

3. The method of claim 1, further comprising the step of: transmitting the received user heart data to a remote server, wherein said determining steps are performed at the remote server.

4. The method of claim 3, wherein the user interface comprises a web portal.

5. The method of claim 1, further comprising the step of further comprising the step of receiving, by a medical professional via the user interface, determined user's risk of a current infection.

6. The method of claim 1, wherein the method is performed at a regular interval for the user by a remote service.

7. The method of claim 1, wherein (i) the sensor is a heart rate sensor, and the user heart data is heart rate sensor data, and/or (ii) the sensor is an electrocardiography sensor, and the user heart data is ECG data.

8. A method for treating a patient's infection, comprising:
receiving, by a medical professional from a user interface, a patient's determined current infection, wherein the patient's current infection is determined by:
receiving, from a sensor of a wearable device worn by the patient, patient heart data;
determining, from the received patient heart data by a pulse rate variability phase acceleration algorithm, the patient's pulse rate variability phase acceleration values for the first time period;
determining, from the received patient heart data by a pulse rate variability phase deceleration algorithm, the patient's pulse rate variability phase deceleration values for the first time period;
determining, by a trained infection risk prediction algorithm using the patient's pulse rate variability phase acceleration values and the patient's pulse rate variability phase deceleration values for the first time period, that the patient is experiencing a current infection; and
providing, to the medical professional via the user interface, the determination that the patient is experiencing a current infection; and
prescribing, by the medical professional in response to the provided patient's risk of a current infection, an antibiotic and/or anti-viral treatment to treat the patient's current infection.

9. The method of claim 8, wherein (i) the sensor is a heart rate sensor, and the user heart data is heart rate sensor data, and/or (ii) the sensor is an electrocardiography sensor, and the user heart data is ECG data.

10. The method of claim 8, wherein the user interface comprises a web portal.

11. A system for reporting a user's predicted risk of infection, comprising:
a wearable device worn by the user, comprising a sensor configured to obtain user heart data;
a trained infection risk prediction algorithm configured to determine the user's risk of a current infection based on the user's pulse rate variability phase acceleration values and the user's pulse rate variability phase deceleration values for the first time period;
a processor configured to: (i) determine, from the received user heart data by a pulse rate variability phase acceleration algorithm, the user's pulse rate variability phase acceleration values for the first time period; (ii) determine, from the received user heart data by a pulse rate variability phase deceleration algorithm, the user's pulse rate variability phase deceleration values for the first time period; and (iii) determine, by the trained infection risk prediction algorithm using the user's pulse rate variability phase acceleration values and the user's pulse rate variability phase deceleration values for the first time period, the user's risk of a current infection; and
a user interface configured to provide the determined user's risk of a current infection.

12. The system of claim 11, wherein the user interface comprises a web portal.

13. The system of claim 11, wherein the received user heart data is transmitted to a remote server.

14. The system of claim 11, wherein the processor performs the claimed steps at a regular interval for the user.

15. The system of claim 11, wherein (i) the sensor is a heart rate sensor, and the user heart data is heart rate sensor data, and/or (ii) the sensor is an electrocardiography sensor, and the user heart data is ECG data.
